(19) United States
(12) Patent Application Publication
(10) Pub. No.: US 2006/0247773 A1
(43) Pub. Date: Nov. 2, 2006

(54) Instrumented implant for diagnostics

(75) Inventor: Carl M. Stamp, Collierville, TN (US)

Correspondence Address: HAYNES AND BOONE, LLP, 901 MAIN ST, SUITE 3100, DALLAS, TX, 75202, US

(73) Assignee: SDGI Holdings, Inc., Wilmington, DE (US)

(21) Appl. No.: 11/118,170

(22) Filed: Apr. 29, 2005

(51) Int. Cl. A61F 2/44

(52) U.S. Cl. 623/17.11

(57) ABSTRACT

A vertebral implant for interposition between first and second vertebral bodies comprises a first component for engaging a vertebral endplate of the first vertebral body and a second component for engaging a vertebral endplate of the second vertebral body. The second component is adapted to articulate with respect to the first component. The implant further includes a first sensor for detecting a first physical parameter and a transmitter coupled to the first sensor. The transmitter is adapted for interposition between the first and second vertebral bodies.

### BACKGROUND

During the past thirty years, technical advances in the design of large joint reconstructive devices has revolutionized the treatment of degenerative joint disease, moving the standard of care from arthrodesis to arthroplasiy. Progress in the treatment of vertebral disc disease, however, has come at a slower pace. Currently, the standard treatment for disc disease remains discectomy followed by vertebral fusion. While this approach may alleviate a patient's present symptoms, accelerated degeneration of the adjacent discs is a frequent consequence of the forces induced by fusion. Thus, reconstructing the degenerated intervertebral disc with a functional disc prosthesis to provide motion and to reduce deterioration of the adjacent discs may be a more desirable treatment option for many patients. An better understanding of the physical parameters experienced by the functional disc prosthesis within the intervertebral disc space may help to improve the design of future prostheses.

### SUMMARY

In one embodiment of the present disclosure, a vertebral implant for interposition between first and second vertebral bodies comprises a first component for engaging a vertebral endplate of the first vertebral body and a second component for engaging a vertebral endplate of the second vertebral body. The second component is adapted to articulate with respect to the first component. The implant further includes a first sensor for detecting a first physical parameter and a transmitter coupled to the first sensor. The transmitter is adapted for interposition between the first and second vertebral bodies.

In another embodiment of the present disclosure, a system for gathering diagnostic data about a patient comprises an implant for interposition between a pair of vertebral bodies. The implant comprises at least two surfaces adapted for sliding engagement with each other. The system further includes at least one sensor component coupled to the implant and at least one transmitter component coupled to the at least one sensor device and adapted for implantation between the pair of vertebral bodies. The system further comprises a power supply component associated with the at least one transmitter and a receiver component adapted to receive a communication from the at least one transmitter component.

In another embodiment, a vertebral implant for interposition between first and second vertebral bodies comprises a vertebral body replacement component for replacing a third vertebral body removed from between the first and second vertebral bodies. The implant further includes at least one sensor for detecting at least one physical parameter and a transmitter coupled to the sensor.

In another embodiment, a diagnostic system for assessing vertebral joint performance comprises a first sensor engaged with a posterior vertebral bone element for detecting a first physical parameter and a transmitter coupled to the first sensor for transmitting data about the first physical parameter. The system further comprises a receiver in communication with the first sensor for receiving the transmitted data about the first physical parameter.

In another embodiment, a method for gathering data on the operation of a vertebral implant comprises implanting an articulated disc between a pair of vertebral bodies. The articulated disc comprises a pair of slidably engaged surfaces and is fitted with a) at least one sensor for detecting at least one physical parameter and b) a transmitter coupled to the at least one sensor for communicating data about the at least one physical parameter. The method further comprises supplying power to the at least one sensor and transmitting the data from the transmitter to a receiver.

### BRIEF DESCRIPTION OF THE DRAWINGS

FIG. 1 is a side view of a vertebral column.

FIG. 2 is a diagram of a diagnostic system according to one embodiment of the present disclosure.

FIG. 3 is an instrumented implant according to one embodiment of the present disclosure.

FIG. 4 is a flowchart of one embodiment of a method of implementing an instrumented implant for diagnostics.

FIG. 5 is an instrumented implant according to another embodiment of the present disclosure.

FIG. 6 is an instrumented implant according to another embodiment of the present disclosure.

FIG. 7 is an instrumented implant according to another embodiment of the present disclosure.

FIG. 8 is an instrumented implant according to another embodiment of the present disclosure.

### DETAILED DESCRIPTION

The present invention relates generally to vertebral reconstructive devices, and more particularly, to instrumented vertebral implants. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

Referring first to FIG. 1, the numeral 10 refers to a vertebral joint which includes an intervertebral disc 12 extending between vertebrae 14, 16. The vertebra 14 includes a vertebral body 14a, a spinous process 14b, and a caudal articular process 14c. The vertebra 16 includes a vertebral body 16a, a spinous process 16b, and a rostral articular process 16c. Another intervertebral disc 18 extends between vertebrae 16 and 20. The disc 12 may be partially or entirely removed and an intervertebral implant 22 may be inserted between the vertebrae 14, 16 to preserve motion within the joint 10. Although the illustration of FIG. 1 generally depicts the vertebral joint 10 as a lumbar vertebral joint, it is understood that the devices, systems, and methods of this disclosure may also be applied to all regions of the vertebral column, including the cervical and thoracic regions. Additionally, although the illustration of FIG. 1 generally depicts an anterior approach for insertion of the implant 22, other approaches including posterior, posterolateral, lateral, and anterolateral are also contemplated. Furthermore, the devices, systems, and methods of this disclosure may be used in non-spinal orthopedic applications.

To better understand the specific physical conditions experienced by a vertebral implant, the implant may be instrumented, or fitted with various diagnostic sensors capable of detecting physical parameters. The parameters may, for example, include pressure, linear displacement, angular displacement, torque, velocity, acceleration, temperature, or pH. The data collected from the various sensors can be used to refine the design of a replacement implant or improve the designs of implants for other patients. For example, understanding forces exerted on the implant and the resulting pressure concentrations within the implant may permit design changes that can reduce the weight of the implant and/or localize material strength though material selection or material thickness.

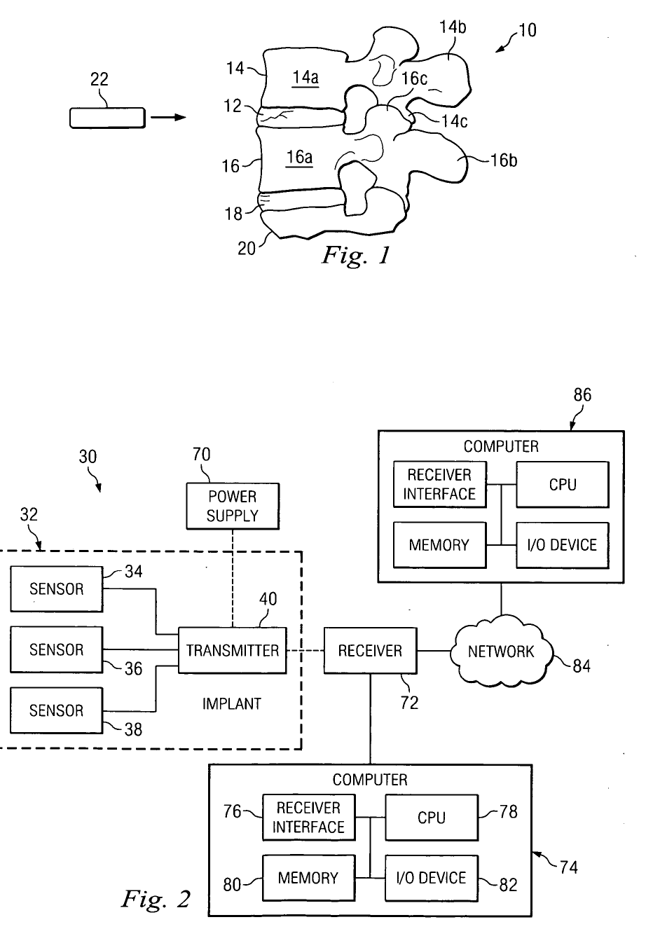

Referring now to FIG. 2, in one embodiment, a system 30 for analyzing physical parameters within a vertebral column may include an implant 32 which may be used as the prosthesis 22 of FIG. 1. The implant 32 may include sensors 34-36 coupled to a biotelemetry transmitter 40. Multiple sensors 34-36 may be used to measure multiple physical parameters simultaneously. For example, sensor 34 may measure shear loading, sensor 36 may measure compressive loading, and sensor 38 may measure motion across the disc space. Although the sensors are depicted as incorporated into the implant, it is understood that sensors may also be located at other positions either internal or external to the patient. Using a sensor both in the implant and in a remote location in the patient may allow the system to capture differential motion, for example.

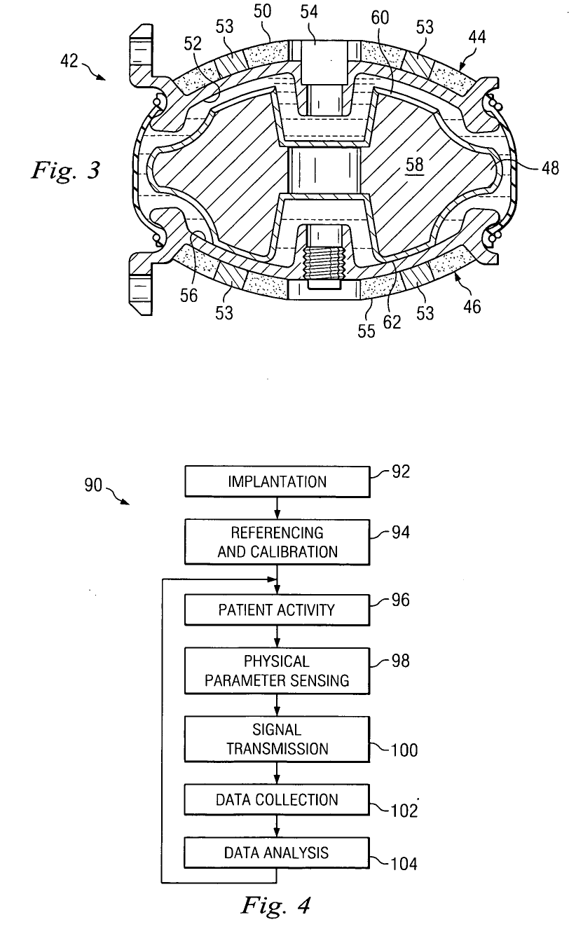

Referring now to FIG. 3, in one embodiment the implant 32 may be an articulated disc implant 42 similar to the implant disclosed in U.S. patent application Ser. No. 09/924,298, entitled “Implantable Joint Prosthesis” and incorporated herein by reference. Although described in more detail in the referenced application, the implant 42 generally includes opposing endplate components 44, 46 between which a central body 48 may articulate. The endplate component 44 includes an exterior surface 50 and an interior articulating surface 52. In this embodiment, the interior surface 52 may be relatively smooth and may have a mirror surface finish. The endplate component 46 may have an exterior surface 55 and an interior articulating surface 56. The interior surface 56 may also be relatively smooth and may have a mirror surface finish. The surfaces 52, 56 may be treated with any of various techniques to improve wear resistance such as ion-implantation, diamond or diamond-like coating, or other methods that make the surface harder than the original surface.

The implant 42 may also include sensors 53, corresponding to sensors 34-38 of the system 30 for detecting physical parameters. The implant 42 may further include a transmitter 54 which may be electrically coupled to the sensors 53. The transmitter 54 may correspond to the transmitter 40 of system 30. It is understood that additional components such as power components, memory components, or a central processing unit (CPU) may be incorporated the implant as needed. The location of the sensors 53 in FIG. 3 is merely exemplary, and it is understood that the sensors may be located at any position in or on the implant 42 to monitor a desired physical parameter. Physical parameters that may be monitored include, for example, pressure, linear displacement, angular displacement, torque, velocity, acceleration, temperature, or pH.

A pressure sensor may, for example, use Wheatstone bridge based strain gauge technology. Alternative pressure sensors may include inductive or capacitive measurement systems. A linear displacement sensor may, for example, use linear variable differential transformer (LVDT) technology to measure linear displacements. Likewise, an angular displacement may, for example, use rotational variable differential transformer (RVDT) technology to measure angular displacement. An acceleration sensor may, for example, include an accelerometer. It is understood that multiple sensors of various types may be used in a single implant to measure different physical parameters.

The central body 48 extends between the interior articulating surfaces 52, 56. The central body 48 may have an inner portion 58 and outer surfaces 60, 62. Although not shown, sensors similar to sensors 53 may be incorporated into the central body 48. The inner portion 58 may be flexible and formed from one or more resilient materials which may have a lower modulus than the outer surfaces. Suitable materials may include polymeric elastomers such as polyolefin rubbers; polyurethanes (including polyetherurethane, polycarbonate urethane, and polyurethane with or without surface modified endgroups); copolymers of silicone and polyurethane with or without surface modified endgroups; silicones; and hydrogels. Polyisobutylene rubber, polyisoprene rubber, neoprene rubber, nitrile rubber, and/or vulcanized rubber of 5-methyl-1,4-hexadiene may also be suitable. In an alternative embodiment, the inner portion 58 may be rigid and formed of any of the materials described below for the outer surfaces or the endplate components.

The outer surfaces 60, 62 of the central body 48 may also be formed of the resilient and flexible materials described above, but in the alternative, they may be modified, treated, coated or lined to enhance the wear resistant and articulating properties of the core component 48. These wear resistant and articulation properties may be provided by cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Polymer materials may also be used including any member of the PAEK family such as PEEK, carbon-reinforced PAEK, or PEKK; polysulfone; polyetherimide; polyimide; UHMWPE; and/or cross-linked UHMWPE. Polyolefin rubbers, polyurethanes, copolymers of silicone and polyurethane, and hydrogels may also provide wear resistance and articulation properties. Wear resistant characteristics may also or alternatively be provided to the outer surfaces 60, 62 by modifications such as cross-linking and metal ion implantation.

The endplate components 44, 46 may be formed of any suitable biocompatible material including metals such as cobalt-chromium alloys, titanium alloys, nickel titanium alloys, and/or stainless steel alloys. Ceramic materials such as aluminum oxide or alumnia, zirconium oxide or zirconia, compact of particulate diamond, and/or pyrolytic carbon may be suitable. Polymer materials may also be used, including any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE.

The exterior surfaces 50, 55 may include features or coatings (not shown) which enhance the fixation of the implanted prosthesis. For example, the surfaces may be roughened such as by chemical etching, bead-blasting, sanding, grinding, serrating, and/or diamond-cutting. All or portions of the exterior surfaces 50, 55 may receive a coating of a metallic substance which may be applied by sintering or by a spray coating such as a plasma spray. All or a portion of the exterior surfaces 50, 55 may also be coated with a biocompatible and osteoconductive material such as hydroxyapatite (HA), tricalcium phosphate (TCP), and/or calcium carbonate to promote bone in growth and fixation. Alternatively, osteoinductive coatings, such as proteins from transforming growth factor (TGF) beta superfamily, or bone-morphogenic proteins, such as BMP2 or BMP7, may be used. Other suitable features may include spikes for initial fixation; ridges or keels to prevent migration in the lateral and anterior direction, for example; serrations or diamond cut surfaces; fins; posts; and/or other surface textures.

Referring again to FIG. 2, the system 30 may further include a power supply unit 70 associated with the transmitter. Although the power supply 70 is depicted as external to the implant 32, it is understood that in some embodiments all or portions of the power supply may be incorporated into the implant. In one embodiment, the power supply may include a battery pack and a separate radio frequency (RF) signal generator. The battery pack may be coupled to the implant 32 and implanted in the patient. The RF generator may be located externally of the patient and can be used to selectively activate the sensors and transmitter. The battery pack may, for example, power a switch for the sensors and transmitter, and the RF signal generator may activate the switch. An alternative to a battery based power supply unit may be an inductive power system. The sensors and the transmitter may be powered inductively by, for example, an inductive coil fitted externally of the patient on a cervical collar, in the case of a cervical implant. The inductive coil may be located in a torso belt in the case of a lumbar implant.

The system 30 may further include a receiver 72 in communication with the transmitter 40. The transmitter 40 and the receiver 72 may communicate data about the physical parameters detected by the sensors 34-38 through the use of RF signals, however alternative wired or wireless techniques may be used. The receiver 72 may monitor and record the RF signals while attached externally to the patient on, for example, a cervical collar in the case of a cervical implant. A torso belt may be used to position the receiver in the case of a lumbar implant.

The receiver 72 may be connected to a computer 74 for processing the received data about the physical parameters detected by the sensors 34-38. The computer 74 may, for example include a receiver interface component 76, a CPU 78, a memory component 80, and an input/output device 82. Although a computer 74 may be directly connected to the receiver 72, the receiver may also or alternatively be connected via a public or private computer network 84, such a private intranet or the public internet, to a remote computer 86. Computer 86 may be configured similarly to the computer 74.

Referring now to FIG. 4, a process 90 for implementing the system 30 of FIG. 2 may begin with the step 92 of implanting the implant 32 into the vertebral column. Using an anterior, posterior, posterolateral, lateral, or anterolateral approach, the desired location of the implant may be accessed and the implant installed. For example, using an anterior approach, the articulated disc implant 42 may be implanted into the vertebral joint 10 in the void created by the removed disc 12 such that the exterior surface 50 engages an endplate of the vertebral body 14 and the exterior surface 55 engages an endplate of the vertebral body 16.

Proceeding now to step 94, after the implant 32 is installed, the sensors and transmitters may be powered and calibration and reference measurements may be recorded. At step 96, the patient may perform an activity such as standing up, bending, walking, or running. At step 98, the sensors 34-38 may detect the physical parameters associated with the performance of the patient's physical activity. Data associated with the physical parameter may be conveyed to the transmitter. At step 100, the transmitter 40 may transmit the physical parameter data to the receiver 72. At step 102, the physical parameter data may be collected by the computer 74 or 86. At step 104, the physical parameter day may be analyzed to evaluate the design and performance of the implant 32. This procedure 90 may be repeated at various stages of the patient's recovery to evaluate the function of the implant 32 and/or to monitor the progression of any degenerations such as adherence problems, bone wear, subsidence, or implant misalignment. Analysis of the physical parameters may suggest revisions that may be made to the implant in situ. Alternatively, the collected data may suggest redesign strategies that may be used to prepare a replacement disc or discs for other patients.

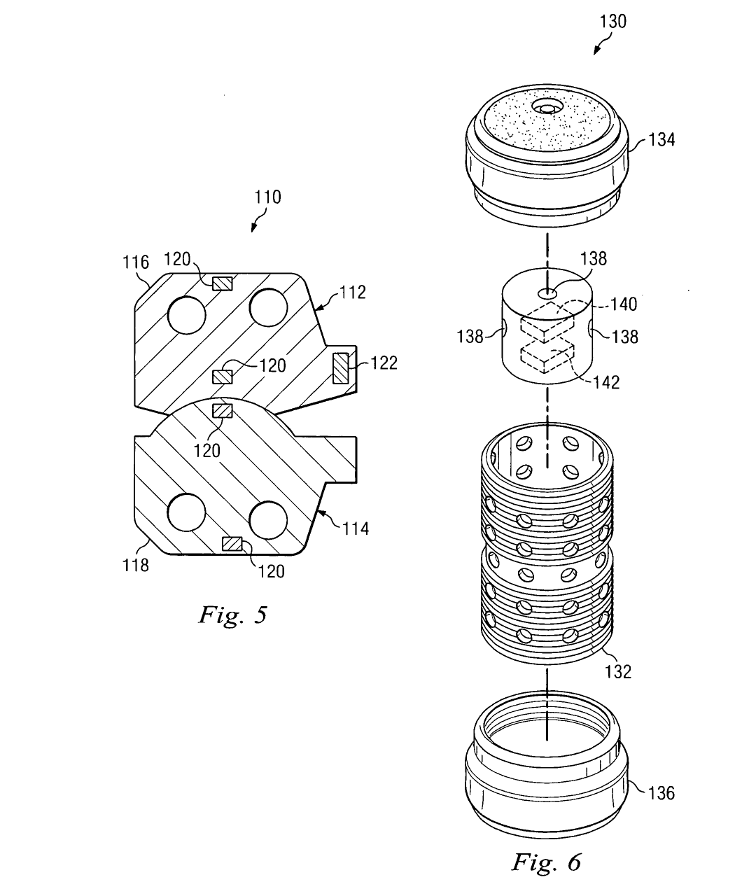

Referring now to FIG. 5, the implant 32 for implantation in the vertebral column may be any of a variety of implants. In this embodiment, an articulating implant 110 includes a first articular component 112 and a second articular component 114. The articular components 112, 114 cooperate to form the articulating joint 110. The articulating joint 110 provides relative pivotal and rotational movement between the adjacent vertebral bodies to maintain or restore motion substantially similar to the normal bio-mechanical motion provided by a natural intervertebral disc. More specifically, the articular components 112, 114 are permitted to pivot relative to one another about a number of axes, including lateral pivotal movement and anterior-posterior pivotal movement. The implant may be formed of any of the materials described above for the components 44, 46 of implant 42. This implant 110 may be similar to the implant described in U.S. Pat. No. 6,740,118, entitled “Intervertebral Prosthetic Joint” which is incorporated herein by reference.

The implant 110 may include fins 116, 118 for penetrating the endplates of the adjacent vertebral bodies to enhance fixation. The implant 110 may also include sensors 120 which may correspond to sensors 34-38 of the system 30 for detecting physical parameters. The implant 110 may further include a transmitter 122 which may be wired or wirelessly coupled to the sensors 120. The transmitter 122 may correspond to the transmitter 40 of system 30. It is understood that additional components such as power components, memory components, a CPU, or additional transmitters may be incorporated the implant as needed. The location of the sensors 120 in FIG. 3 is merely exemplary, and it is understood that the sensors may be located at any position in or on the implant 110 to monitor a desired physical parameter. Physical parameters that may be monitored include, for example, pressure, linear displacement, angular displacement, torque, velocity, acceleration, temperature, or pH. The implant 110 may be implanted and operated using the method 90 of FIG. 4.

Referring now to FIG. 6, an implant 130 may be used following a corpectomy procedure to replace the vertebral body 16 and the adjacent pair of discs 12, 18. In this embodiment, the implant 130 includes a body portion 132 threadedly coupled between two articulating disc implants 134, 136. The implants 134, 136 may be similar to implant 42 described above. The body component may be similar to components described in U.S. Pat. No. 5,702,453, entitled “Adjustable Vertebral Body Replacement” and incorporated herein by reference.

The implant 130 may include sensors 138 which may correspond to sensors 34-38 of the system 30 for detecting physical parameters. The implant 130 may further include a transmitter 140 which may be wired or wirelessly coupled to the sensors 130. The transmitter 140 may correspond to the transmitter 40 of system 30. The implant 130 may also include a power supply 142 which may be a battery electrically connected to the transmitter 140 and or the sensors 138 It is understood that additional components such as power components, memory components, a CPU, or additional transmitters may be incorporated the implant as needed. The power supply 142, the transmitter 140, and/or the sensors 138 may be housed within the body portion 132. The location of the sensors 138 in FIG. 3 is merely exemplary, and it is understood that the sensors may be located at any position in or on the implant 130 to monitor a desired physical parameter. Physical parameters that may be monitored include, for example, pressure, linear displacement, angular displacement, torque, velocity, acceleration, temperature, or pH. The implant 130 may be implanted and operated using the method 90 of FIG. 4.

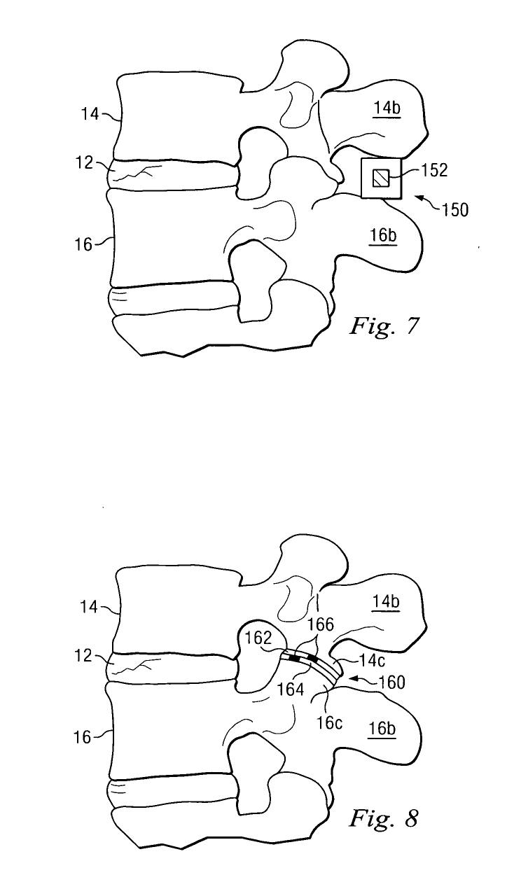

Referring now to FIG. 7, an interspinous implant 150 may be installed between spinous processes 14b, 16b. Portions of the implant 150 may be similar to any number of interspinous implants including U.S. Pat. No. 6,626,944, entitled “Interspinous Prosthesis.” The implant 150 may act as a dampener and/or a distraction mechanism to restore or maintain intervertebral height. The implant 110 may also include a diagnostic package 152 which includes sensors corresponding to sensors 34-38 of the system 30 for detecting physical parameters. The diagnostic package may further include a transmitter which may be wired or wirelessly coupled to the sensors. The transmitter may correspond to the transmitter 40 of system 30. It is understood that additional components such as power components, memory components, a CPU, or additional transmitters may be incorporated the implant 150 as needed. It is understood that the sensors may be located at any position in or on the implant 150 to monitor a desired physical parameter. Physical parameters that may be monitored include, for example, pressure, linear displacement, angular displacement, torque, velocity, acceleration, temperature, or pH. The implant 150 may be implanted using a minimally invasive posterior or posterolateral approach. The method of operation described in steps 94-104 may be used to perform diagnostic testing using the implant 150.

Referring now to FIG. 8, a facet implant 160 may be installed to augment or replace portions of the articular processes 14c, 16c and/or the facet capsule located between the articular processes. The implant 160 may include a pair of articulating surfaces 162, 164 to restore motion to the facet joint. Portions of the implant 160 may be similar to facet replacement or augmentation systems known in the art. The implant 160 may additionally include sensors 166 corresponding to sensors 34-38 of the system 30 for detecting physical parameters. The implant 160 may further include a transmitter which may be wired or wirelessly coupled to the sensors. The transmitter may correspond to the transmitter 40 of system 30. It is understood that additional components such as power components, memory components, a CPU, or additional transmitters may be incorporated the implant 160 as needed. It is understood that the sensors may be located at any position in or on the implant 160 to monitor a desired physical parameter. Physical parameters that may be monitored include, for example, pressure, linear displacement, angular displacement, torque, velocity, acceleration, temperature, or pH. The implant 160 may be implanted using a minimally invasive posterior or posterolateral approach. The method of operation described in steps 94-104 may be used to perform diagnostic testing using the implant 160.

In alternative embodiments, the diagnostic implant may be located within a vertebral body or attached to the posterior bony elements at non-joint locations.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “rostral,” “caudal,” “upper,” and “lower,” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. A vertebral implant for interposition between first and second vertebral bodies comprising:
a first component for engaging a vertebral endplate of the first vertebral body;
a second component for engaging a vertebral endplate of the second vertebral body, wherein the second component is adapted to articulate with respect to the first component;
a first sensor for detecting a first physical parameter; and
a transmitter coupled to the first sensor, wherein the transmitter is adapted for interposition between the first and second vertebral bodies.

2. The vertebral implant of claim 1 wherein the first component is in sliding contact with the second component.

3. The vertebral implant of claim 1 further comprising a central body interposed between the first and second components, wherein the central body is adapted for articulating contact with the first and second components.

4. The vertebral implant of claim 1 wherein the first sensor comprises a linear variable differential transformer.

5. The vertebral implant of claim 1 wherein the first sensor comprises a rotational variable differential transformer.

6. The vertebral implant of claim 1 wherein the first sensor comprises an accelerometer.

7. The vertebral implant of claim 1 wherein the first sensor comprises a pressure transducer.

8. The vertebral implant of claim 1 further comprising a power supply component.

9. The vertebral implant of claim 1 further comprising a second sensor for detecting a second physical parameter.

10. The vertebral implant of claim 1 wherein the first sensor is directly coupled to the first component and the second sensor is directly coupled to the second component.

11. A system for gathering diagnostic data about a patient comprising:
an implant for interposition between a pair of vertebral bodies, the implant comprising at least two surfaces adapted for sliding engagement with each other;
at least one sensor component coupled to the implant;
at least one transmitter component coupled to the at least one sensor device and adapted for implantation between the pair of vertebral bodies;
a power supply component associated with the at least one transmitter; and
a receiver component adapted to receive a communication from the at least one transmitter component.

12. The system of claim 1I1 further comprising:
a computer in communication with the receiver component.

13. The system of claim 12 wherein the computer is in communication with the receiver component via a public network.

14. The system of claim 11 wherein the power supply component is adapted for implantation between the pair of vertebral bodies.

15. The system of claim 14 further comprising a power supply unit located externally of the patient and adapted for energizing the power supply component.

16. The system of claim 14 wherein the power supply component is a battery.

17. The system of claim 11 wherein the communication is transmitted via radio-frequency.

18. The system of claim 11 wherein at least one of the vertebral bodies is a cervical vertebral body and the receiver component is connected to a collar for at least partially encircling the patient's neck.

19. The system of claim 11 wherein at least one of the vertebral bodies is a lumbar vertebral body and the receiver component is connected to a belt for at least partially encircling the patient's torso.

20. The system of claim 11 further comprising:
a remote sensor coupled to the patient's body for measuring differential motion between the remote sensor and the at least one sensor component coupled to the implant.

21. The system of claim 20 wherein the remote sensor is implanted into the patient outside of the vertebral column.

22. The system of claim 20 wherein the remote sensor is attached to the exterior body of the patient.

23. A vertebral implant for interposition between first and second vertebral bodies comprising:
a vertebral body replacement component for replacing a third vertebral body removed from between the first and second vertebral bodies;
at least one sensor for detecting at least one physical parameter; and
a transmitter coupled to the sensor.

24. The vertebral implant of claim 23 further comprising:
at least one articulated vertebral disc replacement component coupled to the vertebral body replacement component.

25. The vertebral implant of claim 23 wherein the at least one sensor is at least one pressure transducer.

26. The vertebral implant of claim 23 wherein the at least one sensor is adapted to measure linear displacement.

27. The vertebral implant of claim 23 wherein the at least one sensor is adapted to measure angular displacement.

28. The vertebral implant of claim 23 wherein the at least one sensor is adapted to measure relative motion between the first and second vertebral bodies.

29. The vertebral implant of claim 23 wherein the at least one sensor is adapted to measure acceleration.

30. A diagnostic system for assessing vertebral joint performance comprising:
a first sensor engaged with a posterior vertebral bone element for detecting a first physical parameter;
a transmitter coupled to the first sensor for transmitting data about the first physical parameter; and
a receiver in communication with the first sensor for receiving the transmitted data about the first physical parameter.

31. The diagnostic system of claim 30 wherein the posterior vertebral bone element is an articular process

32. The diagnostic system of claim 30 wherein the posterior vertebral bone element is a spinous process.

33. The diagnostic system of claim 30 wherein the first sensor is coupled to a facet replacement component and the facet replacement component is engaged with the posterior vertebral bone element.

34. A method for gathering data on the operation of a vertebral implant, the method comprising:
implanting an articulated disc between a pair of vertebral bodies, wherein the articulated disc comprises a pair of slidably engaged surfaces and wherein the articulated disc is fitted with
a) at least one sensor for detecting at least one physical parameter and b) a transmitter coupled to the at least one sensor for communicating data about the at least one physical parameter;
supplying power to the at least one sensor; and
transmitting the data from the transmitter to a receiver.

35. The method of claim 34 wherein the at least one physical parameter is linear displacement.

36. The method of claim 34 wherein the at least one physical parameter is angular displacement.

37. The method of claim 34 wherein the at least one physical parameter is pressure.

38. The method of claim 34 wherein the at least one physical parameter is temperature.

39. The method of claim 34 wherein the at least one physical parameter is acceleration.

40. The method of claim 34 wherein the at least one physical parameter is pH.
